(19) United States
(12) Patent Application Publication
(10) Pub. No.: US 2015/0148944 A1
(43) Pub. Date: May 28, 2015

(54) BODYIMMUNE ENHANCING COMPOSITIONS

(71) Applicant: METAMUNE INC. dba BODYMUNE, Seattle, WA (US)

(72) Inventor: Satyen Chatterjee, Seattle, WA (US)

(21) Appl. No.: 14/555,686

(22) Filed: Nov. 27, 2014

Related U.S. Application Data
(60) Provisional application No. 61/909,918, filed on Nov. 27, 2013.

(51) Int. Cl. G07F 13/06; A61K 36/55; A61K 36/33; A61K 36/889; A61K 36/38; A61K 36/185; A61K 36/815; A61K 36/79; A61K 36/235; A61K 36/48; A61K 45/06; A61K 36/71; A61K 36/31; A61K 36/9066; A61K 36/28; A61K 36/9068; A61K 36/21; G07F 17/00; A61K 9/14

(52) U.S. Cl. CPC G07F 13/06; A61K 9/14; A61K 36/55; A61K 36/33; A61K 36/889; A61K 36/38; A61K 36/185; A61K 36/815; A61K 36/79; A61K 36/235; A61K 36/48; A61K 45/06; A61K 36/71; A61K 36/31; A61K 36/9066; A61K 36/28; A61K 36/9068; A61K 36/21; G07F 17/0092
USPC 700/233; 424/727; 424/489

(57) ABSTRACT

Disclosed herein are method and system for preserving freshness and effectiveness of a composition of natural organic or non-organic ingredients for multiple ailments by stimulating the immune system. Also provided herein are method and system for ordering such compositions via a vending machine or a web interface.

### CROSS-REFERENCE TO RELATED APPLICATIONS

This application claims priority from U.S. provisional application No. 61/909,918, filed on Nov. 27, 2013 and entitled “Body Immune Enhancing Compositions,” which is incorporated herein by reference in its entirety.

### FIELD OF THE INVENTION

The invention disclosed herein generally relates to nutraceutical or pharmaceutical compositions comprising of anti-inflammatory, antioxidants and omegas in a powder form. It also relates to processing methods (e.g., making and packaging) for producing the nutraceutical or pharmaceutical compositions. Also provided are methods for delivering the nutraceutical or pharmaceutical compositions for individualized solution.

### BACKGROUND

Immune disorders and related ailments have long been hurdles to deteriorating health. Treatment of such has been challenging. For example, age-related degradation of human immune system happens mostly gradually. It is difficult for a person to take the initiative to continuously monitor and keep records of his or her health and then to seek help from health professionals, based on one or more health-related changes observed during the self-monitoring process. Often the healthcare professionals will need to conduct additional tests and examinations before prescribing clinical or pharmaceutical treatment. Most of the time, people only seek professional help after some obvious or serious ailment has already taken place.

What is needed in the art are alternative methods to clinical or pharmaceutical treatment that are proven to be safe and effective, which a person can regularly self-administer, as a form of treatment or more ideally as a preventive measure, to improve one's immune health.

### SUMMARY OF THE INVENTION

In one aspect, provided herein is a composition. The composition comprises an anti-inflammatory agent; an anti-oxidant; and an omega acid, where the anti-inflammatory agent, anti-oxidant, and omega vitamin are each in dry or semi-dry powder form.

In some embodiments, at least one of the anti-inflammatory agent, anti-oxidant, and omega acid has a particle size or coarseness in a predetermined range.

In some embodiments, the predetermined range is selected from the group consisting of 300 microns and 800 microns, 300 microns and 750 microns, 300 microns and 710 microns, 300 microns and 630 microns, 300 microns and 600 microns, 300 microns and 560 microns, 300 microns and 500 microns, 300 microns and 450 microns, 300 microns and 425 microns, 300 microns and 355 microns, 300 microns and 315 microns, 425 microns and 800 microns, 425 microns and 750 microns, 425 microns and 710 microns, 425 microns and 630 microns, 425 microns and 600 microns, 425 microns and 560 microns, 425 microns and 500 microns, and 425 microns and 450 microns.

In some embodiments, particles of the anti-inflammatory agent, anti-oxidant, and omega acid in the predetermined range constitutes 80% or more of the total weight of the composition.

In some embodiments, particles of the anti-inflammatory agent, anti-oxidant, and omega acid in the predetermined range constitutes 90% or more of the total weight of the composition.

In some embodiments, the anti-inflammatory agent, anti-oxidant, and omega acid are selected from the group consisting of flaxseed, nopal pad, acai berry fruit, mangosteen fruit, sea buckthorn berry, Goji berry, maqui berry, schisandra berry, fennel seed, fenugreek seed, pomegranate juice powder, sea buckthorn fruit oil, sea buckthorn seed oil, nigella sativa seed, moringa leaf, amla fruit, maca root, turmeric root, burdock root, ginger root, beet juice powder, and combinations thereof.

In one aspect, provided herein is a method of making a composition comprising an anti-inflammatory agent, an anti-oxidant, and an omega acid, all in dry powder form. The method comprises the steps of: grinding at least one of the anti-inflammatory agent, anti-oxidant, and omega acid into particulates, wherein at least 80% or more of the particulates have a size within a predetermined range; providing air-free package of at least one of the anti-inflammatory agent, anti-oxidant, and omega acid; and providing UV protected package of the composition.

In some embodiments, the predetermined range is selected from the group consisting of 300 microns and 800 microns, 300 microns and 750 microns, 300 microns and 710 microns, 300 microns and 630 microns, 300 microns and 600 microns, 300 microns and 560 microns, 300 microns and 500 microns, 300 microns and 450 microns, 300 microns and 425 microns, 300 microns and 355 microns, 300 microns and 315 microns, 425 microns and 800 microns, 425 microns and 750 microns, 425 microns and 710 microns, 425 microns and 630 microns, 425 microns and 600 microns, 425 microns and 560 microns, 425 microns and 500 microns, and 425 microns and 450 microns.

In some embodiments, the anti-inflammatory agent, anti-oxidant, and omega acid are selected from the group consisting of flaxseed, nopal pad, acai berry fruit, mangosteen fruit, sea buckthorn berry, Goji berry, maqui berry, schisandra berry, fennel seed, fenugreek seed, pomegranate juice powder, sea buckthorn fruit oil, sea buckthorn seed oil, nigella sativa seed, moringa leaf, amla fruit, maca root, turmeric root, burdock root, ginger root, beet juice powder, and combinations thereof.

In one aspect, provided herein is a method of ameliorating a medical condition using a composition comprising an anti-inflammatory agent, an anti-oxidant, and an omega acid in dry powder form. The method comprises administering an effective amount of the composition to a subject in need thereof.

In some embodiments, the anti-inflammatory agent, anti-oxidant, and omega acid are selected from the group consisting of flaxseed, nopal pad, acai berry fruit, mangosteen fruit, sea buckthorn berry, Goji berry, maqui berry, schisandra berry, fennel seed, fenugreek seed, pomegranate juice powder, sea buckthorn fruit oil, sea buckthorn seed oil, nigella sativa seed, moringa leaf, amla fruit, maca root, turmeric root, burdock root, ginger root, beet juice powder, and combinations thereof.

In some embodiments, the medical condition is selected from the group consisting of psoriasis, eczema, heartburn/GERD, a heart health condition, an inflammatory condition, general skin condition, hair loss, diseases associated with the inflammation of arteries or various organs of the body, allergy and various diseases due to misplaced immune system, and combinations thereof.

In one aspect, provided herein is a method of providing a composition comprising an anti-inflammatory agent, an anti-oxidant, and an omega acid in dry powder form via a user interface. The method comprises the steps of: receiving a request from a user via the user interface on a networked device; preparing, based on the request, the composition comprising an anti-inflammatory agent, an anti-oxidant, and an omega acid in dry powder form; and dispensing the composition to the user.

In some embodiments, the networked device is a kiosk booth, a vending machine, or a computer web interface.

In some embodiments, the preparing step further comprises the steps of: grinding at least one of the anti-inflammatory agent, anti-oxidant, and omega acid into particulates, wherein at least 80% or more of the particulates have a size within a predetermined range; providing air-free package of at least one of the anti-inflammatory agent, anti-oxidant, and omega acid; and providing UV protected package of the composition.

In some embodiments, the dispensing step comprises a step of releasing the composition to the user from the networked device.

In some embodiments, the dispensing step comprises the step of delivering the composition to the user via courier service.

It will be understood that any embodiment described herein can be used in combination in any aspect of the invention.

### BRIEF DESCRIPTION OF THE DRAWINGS

Those of skill in the art will understand that the drawings, described below, are for illustrative purposes only. The drawings are not intended to limit the scope of the present techniques in any way.

FIG. 1 illustrates an exemplary process.

FIG. 2 illustrates an exemplary process.

### DETAILED DESCRIPTION OF THE INVENTION

Disclosed herein are nutraceutical or pharmaceutical compositions comprising for enhancing body immunity and methods for making thereof. Also disclosed herein are methods of packing and methods of delivery. Further disclosed herein are platforms and methods for dispensing such nutraceutical or pharmaceutical compositions; for example, through a kiosk type vending machine or a small store front or via a web interface.

In one aspect, provided herein are nutraceutical or pharmaceutical compositions comprising of ingredients such as one or more anti-inflammatory agents, one or more antioxidants and one or more omegas in a powder form.

In one aspect, provided herein is a nutraceutical or pharmaceutical composition comprising of ingredients such as one or more anti-inflammatory agents, one or more antioxidants and one or more omegas in a powder form.

In some embodiments, the nutraceutical or pharmaceutical composition comprises Goji berry in powder form as an anti-inflammatory agent. In particular, positive effects of Goji berry (e.g., anti-inflammatory effects) are enhanced by the natural vitamin C present in Acai berry or amla fruit.

In some embodiments, the same ingredient has two or more functionalities. For example, Goji berry can be both an anti-oxidant and an anti-inflammatory agent. See for example, Study from Li X M, et al., 2007, “Effect of the Lycium barbarum polysaccharides on age-related oxidative stress in aged mice,” J Ethnopharmacol 111 (3): 504-511 and articles by Dr. David Jockers at www<dot>naturalnews<dot>com, each of which is hereby incorporated by reference herein in its entirety. It will be understood that concentration or quantity of an ingredient will be adjusted when used mainly for one of the multiple functionalities.

In some embodiments, the nutraceutical or pharmaceutical composition comprises Omega 3, 6, 7 & 9 (in the presence of) from sea buckthorn fruit and seed oils. The presence of Omega 3, 6, 7 & 9 in sea buckthorn fruit and seed oils is also observed to enhance the effectiveness of the antioxidants present in various berries. Trials indicated that a weight ratio of about 4:1 for natural organic sea buckthorn fruit oil to its seed oil gave the best results for inflammation control and joint health. Alternative weight ratios such as 2:1, 2.5:1, 3:1, 3.5:1, 4.5: 1, 5:1, 5.5:1, or 6:1 can also be used. It will be understood that the ratio may need to be adjusted depending on individual conditions; for example, elderly people and young adults may need different ratio combinations. It was also observed that the presence of these oils in the mix enhanced the effectiveness of the nutraceutical or pharmaceutical composition.

In some embodiments, the nutraceutical or pharmaceutical composition is suitable for patients with diabetic conditions or tendencies. For the example, the composition comprises nopal pad, fenugreek seed, amla fruit, turmeric, or combinations thereof. In certain cases, the effect of very low natural sugar content (e.g., about 0.3 to 0.8 grams in daily dosage) from essential fruits and berries is reduced by nopal pad, fenugreek seed, amla fruit, turmeric, which helps to improve diabetic conditions. Nopal pad is a good anti-oxidant but at the same time good for diabetic control. Amla fruit with high natural Vitamin C enhances the anti-inflammatory effectiveness of goji berry, which has natural sugar, and thus reduces the necessary amount of goji berry. In addition, amla fruit has been used over centuries to control diabetic conditions. Fenugreek seed and turmeric root have proven ant-inflammatory effect apart from being good for diabetic conditions.

In some embodiments, the nutraceutical or pharmaceutical composition comprises flaxseed. Flaxseed in addition to its own recognized properties is also observed to act as a protective buffer for small quantities of other valuable ingredients in the blend when they enter the highly acidic stomach environment. Flaxseed in higher volume (for example, around 20% of the total of weight of the nutraceutical or pharmaceutical composition) gives that extra cushion for some ingredients which are in very small quantity, around 0.04 to 0.18 gram, to have time to prove its effects. It's like you don't put wine or spices on a hot flashing pan, you put it on or along with some other ingredients. This can vary depending on the ingredients.

Advantageously, ingredients in the composition disclosed herein have positive synergistic effects when certain combinations of ingredients are taken in combination. Thus, each ingredient taken separately throughout a day is observed not to have the same positive effect. This was established by more than 22 human trials on record by us.

In some embodiments, the composition disclosed herein comprises one or more organic ingredient, such as an organic natural anti-inflammatory reagent. Exemplary organic ingredient include but are not limited to flaxseed, nopal pad, acai berry fruit, mangosteen fruit, sea buckthorn berry, goji berry, maqui berry, schisandra berry, fennel seed, fenugreek seed, pomegranate juice powder, sea buckthorn fruit oil, sea buckthorn seed oil, nigella sativa seed, moringa leaf, amla fruit, maca root, turmeric root, burdock root, ginger root, beet juice powder. Combination of one or more exemplary ingredients are used to treat one or more symptoms of joint and inflammation problems, health problems due to immune imbalance and or inflammation. This Exemplary medical condition include but not are not limited to psoriasis, eczema, heartburn/GERD, a heart health issue and etc.

In one aspect, controlled release of the nutraceutical and pharmaceutical compositions disclosed herein is achieved by controlling grind particles sizes of one or more ingredients. In some embodiments, particular coarseness for each ingredient is used to achieve its controlled release in the body.

For example, to maintain effective buffering quality and in some embodiments, flaxseed coarseness is maintained between 300 microns and 850 microns; for example, using sieve sizes corresponding to American Society for Testing and Materials (ASTM) 2013 mesh sizes No. 20 and No. 50. In some embodiments, flaxseed coarseness is maintained between 300 microns and 800 microns. In some embodiments, flaxseed coarseness is maintained between 300 microns and 750 microns. In some embodiments, flaxseed coarseness is maintained between 300 microns and 710 microns. In some embodiments, flaxseed coarseness is maintained between 300 microns and 630 microns. In some embodiments, flaxseed coarseness is maintained between 300 microns and 600 microns. In some embodiments, flaxseed coarseness is maintained between 300 microns and 560 microns. In some embodiments, flaxseed coarseness is maintained between 300 microns and 500 microns. In some embodiments, flaxseed coarseness is maintained between 300 microns and 450 microns. In some embodiments, flaxseed coarseness is maintained between 300 microns and 425 microns. In some embodiments, flaxseed coarseness is maintained between 300 microns and 355 microns. In some embodiments, flaxseed coarseness is maintained between 300 microns and 315 microns.

In some embodiments, limits of particle size and coarseness are different for different ingredient because how each ingredient gets absorbed and metabolized by the body is different. For example, to avoid sudden and quick impact on the body, coarseness for fenugreek and nigella sativa seeds are maintained at large sizes; for example between 20 to 40 ASTM mesh sizes (or between 425 microns and 800 microns). In some embodiments, coarseness for fenugreek and nigella sativa seeds are maintained between 425 microns and 750 microns. In some embodiments, coarseness for fenugreek and nigella sativa seeds are maintained between 425 microns and 710 microns. In some embodiments, coarseness for fenugreek and nigella sativa seeds are maintained between 425 microns and 630 microns. In some embodiments, coarseness for fenugreek and nigella sativa seeds are maintained between 425 microns and 600 microns. In some embodiments, coarseness for fenugreek and nigella sativa seeds are maintained between 425 microns and 560 microns. In some embodiments, coarseness for fenugreek and nigella sativa seeds are maintained between 425 microns and 500 microns. In some embodiments, coarseness for fenugreek and nigella sativa seeds are maintained between 425 microns and 450 microns.

One would understand that particle size and coarseness control can be applied to any suitable ingredient disclosed herein, including but not limited to for example, two or more ingredients, three or more ingredients, four or more ingredients, five or more ingredients, six or more ingredients, seven or more ingredients, eight or more ingredients, nine or more ingredients, or ten or more ingredients.

In some embodiments, particles within the specified range constitute up to 50% of the total weight of a particular ingredient. In some embodiments, particles within the specified range constitute up to 55% of the total weight of a particular ingredient. In some embodiments, particles within the specified range constitute up to 60% of the total weight of a particular ingredient. In some embodiments, particles within the specified range constitute up to 65% of the total weight of a particular ingredient. In some embodiments, particles within the specified range constitute up to 70% of the total weight of a particular ingredient. In some embodiments, particles within the specified range constitute up to 75% of the total weight of a particular ingredient. In some embodiments, particles within the specified range constitute up to 80% of the total weight of a particular ingredient. In some embodiments, particles within the specified range constitute up to 85% of the total weight of a particular ingredient. In some embodiments, particles within the specified range constitute up to 90% of the total weight of a particular ingredient. In some embodiments, particles within the specified range constitute up to 95% of the total weight of a particular ingredient. Preferably, particles within the specified range constitute up to 90% of the total weight of a particular ingredient.

In one aspect, the nutraceutical compositions disclosed herein are sealed and packaged in UV protective packing (e.g., a UV protected sachet).

For example, freshly ground ingredient or ingredients from sealed containers are mixed in the right proportion in a bulk quantity and each daily dosage is sealed in individual UV protected sachet to preserve the freshness, required to achieve expected efficacy. Preserving the freshness of the combined and individual ingredients is important because UV sensitive organic or non-organic powders lose their potency and effectiveness if not individually put in UV protected sachet. Ingredients from sealed containers or freshly ground ingredients are mixed in the right proportion in a bulk quantity and each daily dosage is then sealed in individual UV protected sachet to preserve the freshness, required to achieve expected efficacy.

Exposure to air also reduces the effectiveness of certain ingredients. In some embodiments, this is overcome by placing fresh ground ingredients in vacuum sealed bags. In some embodiments, a vacuum sealed bag is the same as the UV protected sachet. In some embodiments, fresh ground ingredients are vacuum sealed first before being put into a UV protected sachet. In such embodiments, a UV protected sachet contains multiple packets of vacuum sealed fresh ground ingredients. In some embodiments, when individual ingredients have beneficial synergistic effects when combined together, ingredients are first individually vacuum sealed and are mixed together by a user just before administering the composition. This way, any time-sensitive beneficial synergistic effects take place after the composition has been administered; and therefore, the benefits are maximized.

In this disclosure, the term “a user” is used interchangeably with terms such as “an individual,” “a patient,” “a subject” and etc.

In one aspect, the nutraceutical compositions disclosed herein are administered through the mouth with mixture of compositions and liquid (e.g., a glass of water).

In some embodiments, to maximize the effective absorption process, the ingredients are delivered through the mouth with mixture of the ingredients and liquids, for example a glass of water. The combination of the ingredients may or may not be organic or organic certified. Effectiveness of the ingredient is further enhanced by using Organic certified ingredients.

Some ingredients, for example sea buckthorn seed oil, require to be absorbed by the body mainly through the millions of pores in the mouth and esophagus. The stomach and intestine create a very harsh environment for certain, for example sea buckthorn oil, ingredients when taken in pill or capsule form. In some embodiments, to maximize the effective absorption process, the ingredients are delivered in a powder form through the mouth mixed with a liquid, such as water.

In one aspect, the nutraceutical and pharmaceutical composition as disclosed herein individualized solutions are provided to as individual solutions to general health problems. For example, by offering individualized blends proprietary organic or non-organic powder blends offered to individual users. In some embodiments, the individualized compositions are assigned and then delivered them in sealed UV protected sachet format. All the blends will be mixed and packaged using high quality natural organic or non-organic ingredients in a fully certified, clean and hygienic environment. In some embodiments, individualized blends comprises will be 5-10 ingredients. In some embodiments, the number of ingredients is but may be adjusted higher or lower depending on the combination of ingredients required for individual treatment. In some embodiments, individualized blends comprises 2-5 ingredients; 4-6 ingredients; 5-8 ingredients, 7-10 ingredients, 8-11 ingredients, 9-12 ingredients, 10-15 ingredients, 12-17 ingredients, 16-20 ingredients, 20 or more ingredients, or 25 or more ingredients.

In one aspect, provided herein are platforms and methods for dispensing nutraceutical or pharmaceutical compositions disclosed herein; for example, through a kiosk type vending machine, a small store front or via a web interface.

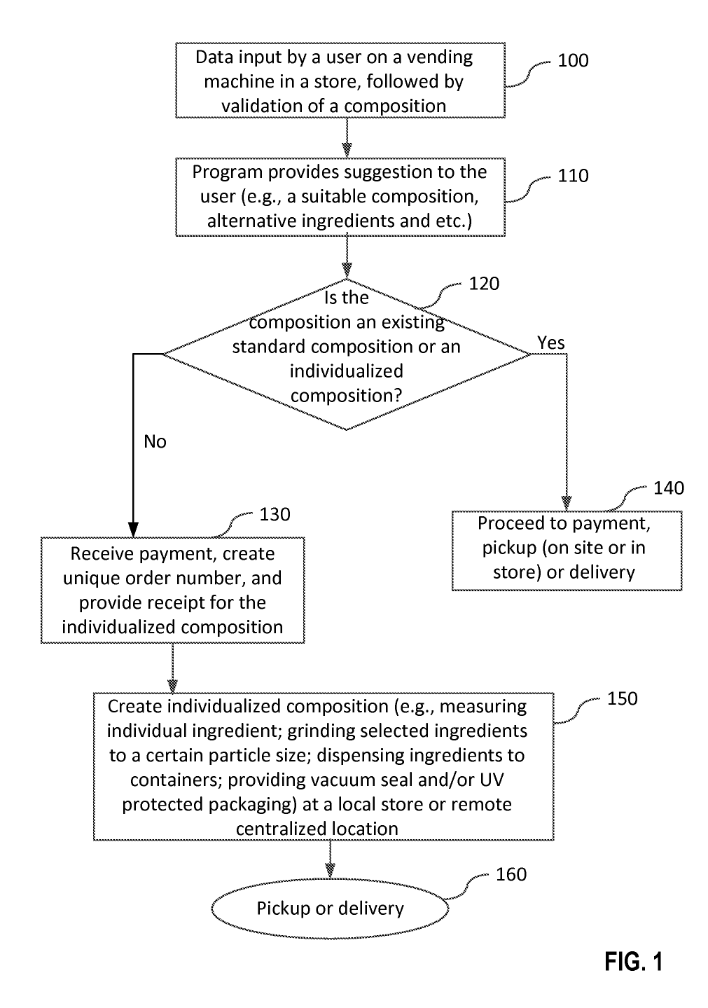

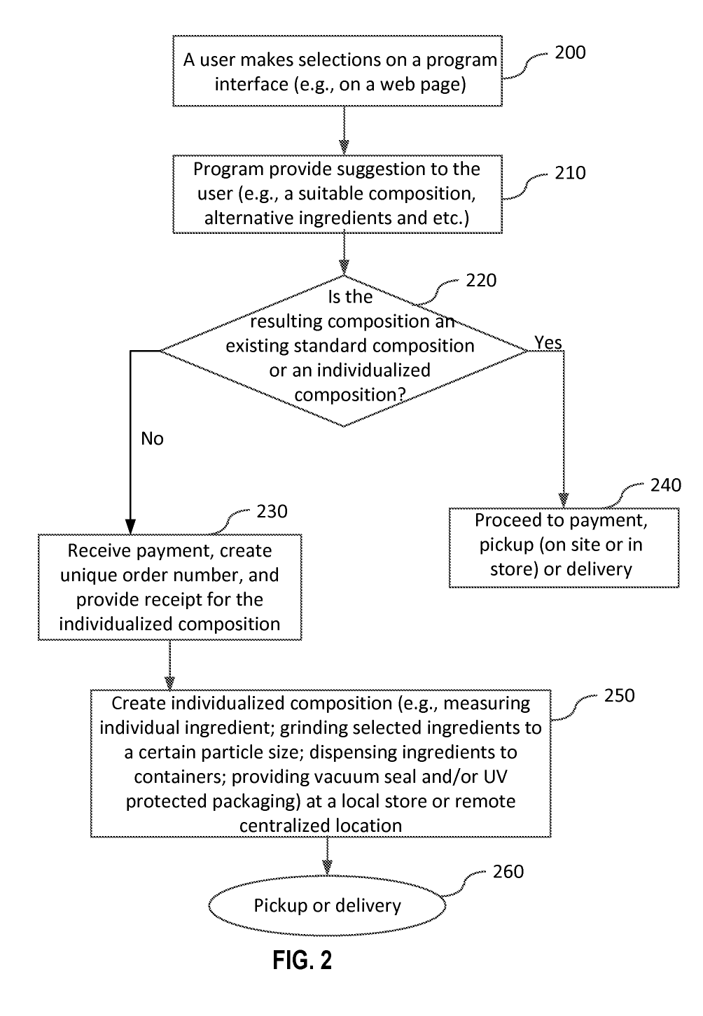

For example, a user can select ingredients to be included in an individualized composition on a touch screen of a network capable vending machine (e.g., FIG. 1). If the machine is located in a local service center, it is possible to have a non-network capable machine. Selection made by a user can be printed and received by staff of the service center, who then processes it locally or transmits it to a remote centralized processing center. Alternatively, such selection can be made on a selection interface via a web browser (e.g., FIG. 2). Here, a typical process is illustrated in an example using a kiosk-type vending machine in a local store, as depicted in FIG. 1. A user inputs data via a touch screen or any data connected device/application or user interface (e.g., step 100). At the end of data entry, the program interface provides suggestions on suitable composition or alternative ingredients (e.g., step 110). A validation process is then initiated to determine whether the ingredient request entered by the user is an existing standard composition or an individualized composition (e.g., 120). If it is the former, the process moves onto a payment step before the standard composition will be delivered either onsite or via a courier service (e.g., step 140).

When a request for an individualized composition is recognized, a user is given a receipt with order number upon payment (e.g., step 130). In some embodiments, on a vending machine with dispensing and packaging capacities, the user can enter the order number. When the machine receives the order, various ingredients represented on the order are accurately measured from various ingredient storage compartments (e.g., ingredient hoppers) (e.g., step 150). In some embodiments, the measured ingredients are further processed to control particle size and coarseness before the ingredient is dispensed into a container, the contents of which will be sealed in UV protected sachets as per the dosage requirements (e.g., step 150). In some embodiments, the quantified and sometimes processed ingredients are put into UV protected packing (e.g., a sachet) with order number attached thereto or printed thereon (e.g., step 150). The processing steps can take place locally in the store or remotely in a centralized location. A finalized composition can be delivered to the user or picked up by the user in a store (e.g., step 160). Optionally, a cross-validation step is performed before the packaged composition is released to a user. For example, the user needs to enter an individualized passcode or receipt number.

For web-based order, a similar process takes place, as illustrated in steps 200-260 in FIG. 2. The main difference is that a user will go to a web portal to select a desired composition (i.e., existing standard composition) or instruct an interface of the type of composition that he or she may need based on certain bodily conditions (e.g., step 200). The payment step is similar to those shown in FIG. 1. At the end, a user has the choice to go to a service center or store for pickup or using a courier service.

In some embodiments, the machine with dispensing and packaging capacities is separate from the vending machine; for example, it is hosted in a physical store or in a factory location. In such embodiments, the order is received at the store or factory. Various ingredients represented on the order received are accurately measured from various ingredient storage compartments (e.g., ingredient hoppers). In some embodiments, the measured ingredients are further process to control particle size and coarseness. Here, more refined processing can take place to precisely control the range of particle size and coarseness. Following processing, the ingredients are either packaged together or individually packaged; e.g., in vacuum sealed bags. In some embodiments as noted above, the bags can provide UV protection. In other embodiments, the vacuum sealed bags are further packaged in one or more UV protected packaging (such as one or more UV sachets). The packaged compositions are then ready for user pickup or delivery. In some embodiments, the user can elect to receive an email or text message notifying them that the ordered composition is ready for pickup or being delivery. For delivery, tracking information can be provided.

In some embodiments, each dedicated service center (store, cafeteria) can have several such kiosks. In some embodiments, the kiosk, through artificial intelligence program and logic, provides a suggested composition with the names of the ingredients based on choices or selections provided by a user. In some embodiments, supplemental needs are determined based on logical steps embedded in the software program interface of the vending machine or web interface. In some embodiments, these logical steps are presented in the form of one or more questionnaires. In some embodiments, the logical steps are written by well-qualified team of doctors. In some embodiments, the logical steps are based on market surveys.

In some embodiments, if a user is allergic to some ingredients, the system can (particle size and coarseness—not correct) provide some alternative ingredients or combinations of alternative ingredients. Otherwise, if no such alternatives are available, no suggestion will be provided. At the end, if available, the individual has the individualized proprietary blend best suited for her/him. It is also possible that no composition can be provided due to the lack of alternative ingredients. In some embodiments, an individual is also offered an alternative from the existing product line. If chosen, the individual can follow the standard purchasing procedure.

If individualized option is chosen, suggested dosage and usage directions would be shown with all the standard disclosures and prices for various options, like the one offered at any store front. Individual can then make the payment there using any available payment methods.

Once the payment is done, the fully automated certified mixing and packaging facility takes over within an appropriate protected enclosure. Machines accurately weighs and picks up the selected ingredients, may be 1 or more, mixes them and then seals the daily dosages in individually sealed UV protected sachets.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### EXAMPLE 1

### Exemplary Ingredients in Powder Form

This examples discloses a combination of ingredients in powder form such as, but not limited to, flaxseed 17-21%, nopal pad 7-12%, pomegranate juice 15-22%, acai berry fruit 4-6%, maqui berry 4-6%, schisandra berry 3-3%, mangosteen fruit 4-7%, sea buckthorn fruit 3-5%, goji berry fruit 5-15%, fenugreek seed 2-3%, fennel seed 0.5-2%, moringa leaf 2-4%, sea buckthorn fruit oil 3-5%, sea buckthorn seed oil 1-2%, maca root 1.5-3%, burdock root 3-5%, amla fruit 2-4%, beet juice powder 2-4%, nigella sativa seed 0.5-1.5%, turmeric root 0.5-1%, ginger root 0.5-1%, can be used to treat one or more symptoms of joint and inflammation problems.

A minimum daily dosage of 6 grams is required to achieve observed benefits. This could be adjusted for various body weights.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the specific number of antigens in a screening panel or targeted by a therapeutic product, the type of antigen, the type of cancer, and the particular antigen(s) specified. Various embodiments of the invention can specifically include or exclude any of these variations or elements.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. A composition, comprising:
an anti-inflammatory agent;
an anti-oxidant; and
an omega acid;
wherein the anti-inflammatory agent, anti-oxidant, and omega acid are in dry/semi-dry powder form.

2. The composition of claim 1, wherein at least one of the anti-inflammatory agent, anti-oxidant, and omega acid has a particle size or coarseness in a predetermined range.

3. The composition of claim 2, wherein the predetermined range is selected from the group consisting of 300 microns and 800 microns, 300 microns and 750 microns, 300 microns and 710 microns, 300 microns and 630 microns, 300 microns and 600 microns, 300 microns and 560 microns, 300 microns and 500 microns, 300 microns and 450 microns, 300 microns and 425 microns, 300 microns and 355 microns, 300 microns and 315 microns, 425 microns and 800 microns, 425 microns and 750 microns, 425 microns and 710 microns, 425 microns and 630 microns, 425 microns and 600 microns, 425 microns and 560 microns, 425 microns and 500 microns, and 425 microns and 450 microns.

4. The composition of claim 2, wherein particles of the anti-inflammatory agent, anti-oxidant, and omega acid in the predetermined range constitutes 80% or more of the total weight of the composition.

5. The composition of claim 2, wherein particles of the anti-inflammatory agent, anti-oxidant, and omega acid in the predetermined range constitutes 90% or more of the total weight of the composition.

6. The composition of claim 1, wherein the anti-inflammatory agent, anti-oxidant, and omega vitamin are selected from the group consisting of flaxseed, nopal pad, acai berry fruit, mangosteen fruit, sea buckthorn berry, Goji berry, maqui berry, schisandra berry, fennel seed, fenugreek seed, pomegranate juice powder, sea buckthorn fruit oil, sea buckthorn seed oil, nigella sativa seed, moringa leaf, amla fruit, maca root, turmeric root, burdock root, ginger root, beet juice powder, and combinations thereof.

7. A method of making a composition comprising an anti-inflammatory agent, an anti-oxidant, and an omega vitamin in dry powder form, comprising:
grinding at least one of the anti-inflammatory agent, anti-oxidant, and omega vitamin into particulates, wherein at least 80% or more of the particulates have a size within a predetermined range;
providing air-free package of at least one of the anti-inflammatory agent, anti-oxidant, and omega vitamin; and
providing UV protected package of the composition.

8. The method of claim 7, wherein the predetermined range is selected from the group consisting of 300 microns and 800 microns, 300 microns and 750 microns, 300 microns and 710 microns, 300 microns and 630 microns, 300 microns and 600 microns, 300 microns and 560 microns, 300 microns and 500 microns, 300 microns and 450 microns, 300 microns and 425 microns, 300 microns and 355 microns, 300 microns and 315 microns, 425 microns and 800 microns, 425 microns and 750 microns, 425 microns and 710 microns, 425 microns and 630 microns, 425 microns and 600 microns, 425 microns and 560 microns, 425 microns and 500 microns, and 425 microns and 450 microns.

9. The method of claim 7, wherein the anti-inflammatory agent, anti-oxidant, and omega vitamin are selected from the group consisting of flaxseed, nopal pad, acai berry fruit, mangosteen fruit, sea buckthorn berry, Goji berry, maqui berry, schisandra berry, fennel seed, fenugreek seed, pomegranate juice powder, sea buckthorn fruit oil, sea buckthorn seed oil, nigella sativa seed, moringa leaf, amla fruit, maca root, turmeric root, burdock root, ginger root, beet juice powder, and combinations thereof.

10. A method of ameliorating a medical condition using a composition comprising an anti-inflammatory agent, an anti-oxidant, and an omega acid in dry powder form, comprising:
administering an effective amount of the composition to a subject in need thereof.

11. The method of claim 10, wherein the anti-inflammatory agent, anti-oxidant, and omega acid selected from the group consisting of flaxseed, nopal pad, acai berry fruit, mangosteen fruit, sea buckthorn berry, Goji berry, maqui berry, schisandra berry, fennel seed, fenugreek seed, pomegranate juice powder, sea buckthorn fruit oil, sea buckthorn seed oil, nigella sativa seed, moringa leaf, amla fruit, maca root, turmeric root, burdock root, ginger root, beet juice powder, and combinations thereof.

12. The method of claim 10, wherein the medical condition is selected from the group consisting of psoriasis, eczema, heartburn/GERD, a heart health condition, an inflammatory condition, general skin condition, hair loss, diseases associated with the inflammation of arteries or various organs of the body, allergy and various diseases due to misplaced immune system, and combinations thereof.

13. A method of providing a composition comprising an anti-inflammatory agent, an anti-oxidant, and an omega acid in dry powder form via a user interface, comprising:
receiving a request from a user via the user interface on a networked device;
preparing, based on the request, the composition comprising an anti-inflammatory agent, an anti-oxidant, and an omega acid in dry powder form; and
dispensing the composition to the user.

14. The method of claim 13, wherein the networked device is a kiosk booth, a vending machine, or a computer web interface.

15. The method of claim 13, wherein the preparing step further comprises:
grinding at least one of the anti-inflammatory agent, anti-oxidant, and omega acid into particulates, wherein at least 80% or more of the particulates have a size within a predetermined range;
providing air-free package of at least one of the anti-inflammatory agent, anti-oxidant, and omega acid; and
providing UV protected package of the composition.

16. The method of claim 13, wherein the dispensing step comprises:
releasing the composition to the user from the networked device.

17. The method of claim 13, wherein the dispensing step comprises:
delivering the composition to the user via courier service.
